(19) United States
(12) Patent Application Publication
(10) Pub. No.: US 2012/0115833 A1
(43) Pub. Date: May 10, 2012

(54) METHOD OF TREATING HEPATOCELLULAR CARCINOMA

(75) Inventors: Walter Berger, Vienna (AT); Petra Heffeter, Vienna (AT); Bernhard Keppler, Vienna (AT); Hooshmand Sheshbaradaran, Hoboken, NJ (US)

(73) Assignee: NIIKI PHARMA INC., Hoboken, NJ (US)

(21) Appl. No.: 13/274,363

(22) Filed: Oct. 17, 2011

Related U.S. Application Data
(63) Continuation of application No. PCT/US2010/031591, filed on Apr. 19, 2010.
(60) Provisional application No. 61/170,534, filed on Apr. 17, 2009.
(60) Provisional application No. 61/266,926, filed on Dec. 4, 2009.

(51) Int. Cl. A61K 31/555; A61P 35/00

(52) U.S. Cl. 514/186

(57) ABSTRACT

A therapeutic method for treating hepatocellular carcinoma is disclosed comprising administering to a patient in need of treatment a ruthenium complex salt.

### CROSS-REFERENCE TO RELATED PATENT APPLICATIONS

This application is a continuation of PCT/US2010/031591 filed on Apr. 19, 2010, which is entitled to the priority of U.S. Provisional Application No. 61/170,534 filed on Apr. 17, 2009, and U.S. Provisional Application No. 61/266,926 filed on Dec. 4, 2009, the contents of all of which are incorporated by reference herein in their entirety.

### FIELD OF THE INVENTION

The present invention generally relates to methods for treating cancer, and particularly to a method of treating hepatocellular carcinoma.

### BACKGROUND OF THE INVENTION

Primary liver cancer is one of the most common forms of cancer in the world. There are two main types of liver cancer. Hepatocellular carcinoma (HCC, also known as malignant hepatoma) and cholangiocellular carcinoma. HCC is the most common form of primary liver cancer, and develops within the hepatocyte. HCC occurs mostly in men and patients that suffer from cirrhosis. In contrast, cholangiocellular carcinoma or bile duct cancer develops in the small bile ducts within the liver. This type of cancer is more common among women.

Treatment options for hepatocellular carcinoma have been limited, especially in the case of advanced or recurrent hepatocellular carcinoma. Surgery and radiation therapy are options for early stage liver cancer, but not very effective for advanced or recurrent hepatocellular carcinoma. Systematic chemotherapies have not been particularly effective, and there are a very limited number of drugs available for use. The recently approved kinase inhibitor sorafenib has been shown to be effective in treating hepatocellular carcinoma. However, it can slow or stop advanced liver cancer from progressing for only a few months longer than without treatment. Indeed, in a Spanish phase III clinical trial in late stage HCC patients with well preserved liver function, it only added an average of two months to the lifespan.

Sodium trans-[tetrachlorobis(1H-indazole)ruthenate(III)] has been shown to be effective in killing tumor cells in colon caner cell lines SW480 and HT29. Kapitza et al., J. Cancer Res. Clin. Oncol., 131(2):101-10 (2005). However, it is not known whether it would be effective in treating hepatocellular carcinoma.

### SUMMARY OF THE INVENTION

It has now been discovered that the compound sodium trans-[tetrachlorobis(1H-indazole)ruthenate(III)] is especially effective in treating hepatocellular carcinoma. It has also been surprisingly discovered that the compound sodium trans-[terachlorobis(1H-indazole)ruthenate(III)] is equally effective in hepatocellular carcinoma cell lines both sensitive and insensitive to drugs such as sorafenib, doxorubicin, cisplatin, oxaliplatin, and 5-FU. Accordingly, in a first aspect, the present invention provides a method of treating hepatocellular carcinoma, which comprises treating a patient identified as having hepatocellular carcinoma, with a therapeutically effective amount of sodium trans-[tetrachlorobis(1H-indazole)ruthenate(III)].

In a second aspect, the present invention provides a method of preventing or delaying the onset of hepatocellular carcinoma, comprising administering to a patient identified to be in need of prevention, or delaying the onset, of hepatocellular carcinoma a prophylatically effective amount of sodium trans-[terachlorobis(1H-indazole)ruthenate(III)].

The present invention further provides use of sodium trans-[terachlorobis(1H-indazole)ruthenate(III)] for the manufacture of a medicament useful for treating, preventing or delaying the onset of hepatocellular carcinoma.

In yet another aspect, the present invention provides a method of treating refractory or resistant hepatocellular carcinoma comprising identifying a patient having refractory hepatocellular carcinoma and treating the patient with a therapeutically effective amount of sodium trans-[tetrachlorobis(1H-indazole)ruthenate(III)]. In specific embodiments, the patient has a hepatocellular carcinoma that is refractory to a treatment comprising one or more drugs selected from the group consisting of sorafenib, regorafenib, doxorubicin, cisplatin, carboplatin, oxaliplatin, 5-FU and capecitabine.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a graph showing the cytotoxicity of sodium salt of trans-[tetrachlorobis(1H-indazole)ruthenate(III)] in different hepatocellular carcinoma cell lines;

FIG. 2 is a graph showing the activity of sodium salt of trans-[tetrachlorobis(1H-indazole)ruthenate(III)] in inducing apoptosis in Hep3B cell line;

FIG. 3 is a gel image showing that apoptotic cell death induced by sodium trans-[tetrachlorobis(1H-indazole)ruthenate(III)] is further characterized by PARP cleavage;

FIG. 4 is a plot showing that treatment with sodium trans-[terachlorobis(1H-indazole)ruthenate(III)] was found to significantly reduce DNA synthesis rate measured by ³H-thymidine incorporation;

FIG. 5 is a plot showing the activity of sodium trans-[terachlorobis(1H-indazole)ruthenate(III)] (“Test Drug”) against xenografted hepatocellular carcinoma (from Hep3B cells) in female SCID mice as compared to treatment with sorafenib;

FIG. 6 is a plot showing that sodium trans-[terachlorobis(1H-indazole)ruthenate(III)] (“Test Drug”) led to a significant increase in life span of female SCID mice xenografed with hepatocellular carcinoma formed from Hep3B cells.

### DETAILED DESCRIPTION OF THE INVENTION

The present invention is at least in part based on the discovery that the compound sodium trans-[terachlorobis(1H-indazole)ruthenate(III)] is especially effective in treating hepatocellular carcinoma. Accordingly, in accordance with a first aspect of the present invention, a method is provided for treating hepatocellular carcinoma (or malignant hepatoma). Specifically, the method comprises treating a patient having hepatocellular carcinoma with a therapeutically effective amount of trans-[tetrachlorobis(1H-indazole)ruthenate(III)] or a pharmaceutically acceptable salt thereof, such as an alkali metal salt of trans-[tetrachlorobis(1H-indazole)ruthenate(III)] (e.g., sodium trans-[tetrachlorobis(1H-indazole)ruthenate(III)] or potassium trans-[terachlorobis(1H-indazole)ruthenate(III)]) and indazolium trans-[tetrachlorobis(1H-indazole)ruthenate(III)]. That is, the present invention is directed to the use of trans-[terachlorobis(1H-indazole)ruthenate(III)] or a pharmaceutically acceptable salt thereof (an alkali metal salt or indazolium salt) for the manufacture of medicaments for treating hepatocellular carcinoma in patients identified or diagnosed as having hepatocellular carcinoma.

In the various embodiments of this aspect of the present invention, the treatment method optionally also comprises a step of diagnosing or identifying a patient as having hepatocellular carcinoma. The identified patient is then treated with or administered with a therapeutically effective amount of a pharmaceutically acceptable salt of trans-[tetrachlorobis(1H-indazole)ruthenate(III)] such as an alkali metal salt of trans-[tetrachlorobis(1H-indazole)ruthenate(III)] (e.g., sodium trans-[terachlorobis(1H-indazole)ruthenate(III)] or potassium trans-[tetrachlorobis(1H-indazole)ruthenate(III)]). Hepatocellular carcinoma can be diagnosed in any conventional diagnostic methods known in the art including ultrasound, CT scan, MRI, alpha-fetoprotein testing, des-gamma carboxyprothrombin screening, and biopsy.

In addition, it has also been surprisingly discovered that the compound sodium trans-[terachlorobis(1H-indazole)ruthenate(III)] is equally effective in hepatocellular carcinoma cell lines both sensitive and insensitive to drugs such as sorafenib, doxorubicin, cisplatin, oxaliplatin, and 5-FU. Accordingly, the present invention also provides a method of treating refractory hepatocellular carcinoma or hepatocellular carcinoma comprising treating a patient identified as having refractory hepatocellular carcinoma with a therapeutically effective amount of trans-[tetrachlorobis(1H-indazole)ruthenate(III)] or a pharmaceutically acceptable salt thereof such as indazolium salt or an alkali metal salt of trans-[terachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]). In specific embodiments, the patient has a hepatocellular carcinoma that is refractory to a treatment comprising one or more drugs selected from the group consisting of sorafenib, regorafenib, anthracyclines (e.g., doxorubicin, daunorubicin, epirubicin, idarubicin), platinum agents (e.g., cisplatin, carboplatin, oxaliplatin, picoplatin), 5-FU and capecitabine. That is, the present invention is also directed to the use of an alkali metal salt of trans-[terachlorobis(1H-indazole)ruthenate(III)] (e.g., sodium trans-[tetrachlorobis(1H-indazole)ruthenate(III)] or potassium trans-[terachlorobis(1H-indazole)ruthenate(III)]) for the manufacture of medicaments for treating refractory hepatocellular carcinoma, e.g., a hepatocellular carcinoma refractory to one or more drugs chosen from sorafenib, regorafenib, anthracyclines (e.g., doxorubicin, daunorubicin, epirubicin, idarubicin), platinum agents (cisplatin, carboplatin, oxaliplatin, picoplatin), 5-FU and capecitabine.

The term “refractory hepatocellular carcinoma,” as used herein refers to hepatocellular carcinoma that either fails to respond favorably to an antineoplastic treatment that does not include trans-[terachlorobis(1H-indazole)ruthenate(III)] or a pharmaceutically acceptable salt thereof, or alternatively, recurs or relapses after responding favorably to an antineoplastic treatment that does not include trans-[tetrachlorobis(1H-indazole)ruthenate(III)] or a pharmaceutically acceptable salt thereof. Accordingly, “a hepatocellular carcinoma refractory to a treatment” as used herein means a hepatocellular carcinoma that fails to respond favorably to, or resistant to, the treatment, or alternatively, recurs or relapses after responding favorably to the treatment.

Thus, in some embodiments, in the method of the present invention, sodium trans-[tetrachlorobis(1H-indazole)ruthenate(III)] is used to treat hepatocellular carcinoma patients having a tumor that exhibits resistance to a treatment comprising one or more drugs selected from the group consisting of sorafenib, regorafenib, anthracyclines (e.g., doxorubicin, daunorubicin, epirubicin, idarubicin), platinum agents (e.g., cisplatin, carboplatin, oxaliplatin, picoplatin), 5-FU and capecitabine. In other words, the method is used to treat a hepatocellular carcinoma patient having previously been treated with a treatment regimen that includes one or more drugs selected from the group consisting of sorafenib, regorafenib, anthracyclines (e.g., doxorubicin, daunorubicin, epirubicin, idarubicin), platinum agents (e.g., cisplatin, carboplatin, oxaliplatin, picoplatin), 5-FU, tegafur and capecitabine, and whose hepatocellular carcinoma was found to be non-responsive to the treatment regimen or have developed resistance to the treatment regimen. In other embodiments, the method is used to treat a hepatocellular carcinoma patient previously treated with a treatment comprising one or more drugs selected from the group consisting of sorafenib, regorafenib, anthracyclines (e.g., doxorubicin, daunorubicin, epirubicin, idarubicin), platinum agents (e.g., cisplatin, carboplatin, oxaliplatin, picoplatin), 5-FU and capecitabine, but the hepatocellular carcinoma has recurred or relapsed, that is, a hepatocellular carcinoma patient who has previously been treated with one or more such drugs, and whose cancer was initially responsive to the previously administered one or more such drugs, but was subsequently found to have relapsed. In specific embodiments, sodium trans-[terachlorobis(1H-indazole)ruthenate(III)] is used to treat hepatocellular carcinoma patients previously treated with sorafenib or regorafenib, i.e., who have a tumor that exhibits resistance to, or relapsed after a treatment including, sorafenib or regorafenib. In other specific embodiments, sodium trans-[tetrachlorobis(1H-indazole)ruthenate(III)] is used to treat hepatocellular carcinoma patients previously treated with doxorubicin, i.e., who have a hepatocellular carcinoma that exhibits resistance to, or relapsed after a treatment including, doxorubicin. In yet other specific embodiments, sodium trans-[tetrachlorobis(1H-indazole)ruthenate(III)] is used to treat hepatocellular carcinoma patients previously treated with a platinum cytotoxic agent (e.g., cisplatin, carboplatin, oxaliplatin, picoplatin), i.e., who have a hepatocellular carcinoma that exhibits resistance to, or relapsed after a treatment including, a platinum cytotoxic agent (e.g., cisplatin, carboplatin, picoplatin, oxaliplatin, or picoplatin). In still other specific embodiments, sodium trans-[tetrachlorobis(1H-indazole)ruthenate(III)] is used to treat hepatocellular carcinoma patients previously treated with 5-FU or a prodrug thereof (e.g., tegafur or capecitabine or S1), i.e., who have a hepatocellular carcinoma that exhibits resistance to, or relapsed after a treatment including, 5-FU or or a prodrug thereof (e.g., tegafur or capecitabine or S1).

To detect a refractory hepatocellular carcinoma, patients undergoing initial treatment can be carefully monitored for signs of resistance, non-responsiveness or recurring hepatocellular carcinoma. This can be accomplished by monitoring the patient's cancer's response to the initial treatment which, e.g., may includes one or more drugs selected from the group consisting of sorafenib, regorafenib, doxorubicin, daunorubicin, epirubicin, idarubicin, cisplatin, carboplatin, oxaliplatin, picoplatin, 5-FU, tegafur and capecitabine. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst. 92:205-216 (2000).

In accordance with yet another aspect of the present invention, a method is provided for preventing or delaying the onset of hepatocellular carcinoma (or hepatocellular carcinoma), or preventing or delaying the recurrence of hepatocellular carcinoma, which comprises treating a patient in need of the prevention or delay with a prophylatically effective amount of a pharmaceutically acceptable salt of trans-[tetrachlorobis(1H-indazole)ruthenate(III)] such as an alkali metal salt of trans-[tetrachlorobis(1H-indazole)ruthenate(III)] (e.g., sodium trans-[terachlorobis(1H-indazole)ruthenate(III)] or potassium trans-[tetrachlorobis(1H-indazole)ruthenate(III)]).

It is now known that people with hepatitis B or hepatitis C infection, or having cirrhosis have an increased risk of developing hepatocellular carcinoma. In addition, people who have acute and chronic hepatic porphyrias (acute intermittent porphyria, porphyria cutanea tarda, hereditary coproporphyria, variegate porphyria) and tyrosinemia type I are also at an increased risk of for developing hepatocellular carcinoma. These people can all be candidates for the method of present invention for preventing or delaying the onset of hepatocellular carcinoma using a prophylatically effective amount of, a pharmaceutically acceptable salt of trans-[tetrachlorobis(1H-indazole)ruthenate(III)] such as an alkali metal salt of trans-[terachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]). In addition, patients with a family history of hepatocellular carcinoma can also be identified for the application of the present method of preventing or delaying the onset of hepatocellular carcinoma.

For purposes of preventing or delaying the recurrence of hepatocellular carcinoma, hepatocellular carcinoma patients who have been treated and are in remission or in a stable or progression free state may be treated with a prophylatically effective amount of a pharmaceutically acceptable salt of trans-[tetrachlorobis(1H-indazole)ruthenate(III)] such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) to effectively prevent or delay the recurrence or relapse of hepatocellular carcinoma.

As used herein, the phrase “treating . . . with . . . ” or a paraphrase thereof means administering a compound to the patient or causing the formation of a compound inside the body of the patient.

In accordance with the method of the present invention, hepatocellular carcinoma can be treated with a therapeutically effective amount of a pharmaceutically acceptable salt of trans-[terachlorobis(1H-indazole)ruthenate(III)] such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) alone as a single agent, or alternatively in combination with one or more other anti-cancer agents.

Alkali metal salts of trans-[terachlorobis(1H-indazole)ruthenate(III)] can be made in any methods known in the art. For example, PCT Publication No. WO/2008/154553 discloses an efficient method of making sodium trans-[tetrachlorobis(1H-indazole)ruthenate(III)].

The pharmaceutical compounds such as sodium trans-[terachlorobis(1H-indazole)ruthenate(III)] can be administered through intravenous injection or any other suitable means at an amount of from 0.1 mg to 1000 mg per kg of body weight of the patient based on total body weight. The active ingredients may be administered at once, or may be divided into a number of smaller doses to be administered at predetermined intervals of time, e.g., once daily or once every two days. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with the present invention, it is provided a use of a pharmaceutically acceptable salt of trans-[tetrachlorobis(1H-indazole)ruthenate(III)] such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) for the manufacture of a medicament useful for treating hepatocellular carcinoma. The medicament can be, e.g., in an injectable form, e.g., suitable for intravenous, intra-arterial, intradermal, or intramuscular administration. Injectable forms are generally known in the art, e.g., in buffered solution or suspension.

In addition, a pharmaceutically acceptable salt of trans-[terachlorobis(1H-indazole)ruthenate(III)] such as an alkali metal salt of trans-[terachlorobis(1H-indazole)ruthenate(III)] can also be used in chemoembolization, in which the drug is administered directly into tumor, while an embolizing agent is used to block the blood supply to the tumor thereby trapping the drug within the tumor. For example, lipiodol (iodized oil) and ¹³¹I-lipidol are embolizing agents suitable for use with an alkali metal salt of trans-[terachlorobis(1H-indazole)ruthenate(III)] in chemoembolization. Other useful embolizing agents include gelatin (e.g. GelFoam) or degradable starch microspheres of defined size ranges. These embolizing agents can be given via intrahepatic artery along with an alkali metal salt of trans-[terachlorobis(1H-indazole)ruthenate(III)] via the same route, known as “hepatic artery transcatheter treatments.”

Thus, the method of the present invention also provides a method for treating hepatocellular carcinoma, preventing or delaying the onset of refractory or recurrent hepatocellular carcinoma comprising administering to a patient a pharmaceutically acceptable salt of trans-[terachlorobis(1H-indazole)ruthenate(III)] such as an alkali metal salt of trans-[terachlorobis(1H-indazole)ruthenate(III)] and an embolizing agent. The alkali metal salt of trans-[terachlorobis(1H-indazole)ruthenate(III)] and embolizing agent such as lipiodol (iodized oil), ¹³¹I-lipidol and gelatin can be administered by hepatic artery injection or intraarterial infusion. Administration of lipiodol or ¹³¹I-lipidol for treating hepatocellular carcinoma is generally known in the art.

In accordance with another aspect of the present invention, a pharmaceutical kit is provided comprising in a container a unit dosage form of a pharmaceutically acceptable salt of trans-[terachlorobis(1H-indazole)ruthenate(III)] such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]), and optionally instructions for using the kit in the methods in accordance with the present invention, e.g., treating, preventing or delaying the onset of hepatocellular carcinoma, or preventing or delaying the recurrence of hepatocellular carcinoma, or treating refractory hepatocellular carcinoma. As will be apparent to a skilled artisan, the amount of a therapeutic compound in the unit dosage form is determined by the dosage to be used on a patient in the methods of the present invention. In the kit, a pharmaceutically acceptable salt of trans-[terachlorobis(1H-indazole)ruthenate(III)] such as an alkali metal salt of trans-[tetrachlorobis(1H-indazole)ruthenate(III)] (e.g., sodium trans-[tetrachlorobis(1H-indazole)ruthenate(III)] or potassium trans-[terachlorobis(1H-indazole)ruthenate(III)]) can be in lyophilized form in an amount of, e.g., 25 mg, in an ampoule. In the clinic, the lyophilized form can be dissolved and administered to a patient in need of the treatment in accordance with the present invention. In addition, the kit optionally further includes a unit dose form of an embolizing agent such as lipiodol (iodized oil) or ¹³¹I-lipidol, which can be, e.g., in vials each having 1 to 1000 ml of lipiodol (iodized oil) or ¹³¹I-lipidol or gelatine or microspheres. In addition, the present invention also provides a pharmaceutical composition comprising a therapeutically effective amount of a pharmaceutically acceptable salt of trans-[terachlorobis(1H-indazole)ruthenate(III)] such as an alkali metal salt of trans-[terachlorobis(1H-indazole)ruthenate(III)] (e.g., sodium trans-[tetrachlorobis(1H-indazole)ruthenate(III)] or potassium trans-[terachlorobis(1H-indazole)ruthenate(III)]) in admixture with an embolizing agent (e.g., lipiodol (iodized oil) or ¹³¹I-lipidol, or gelatine).

### EXAMPLE

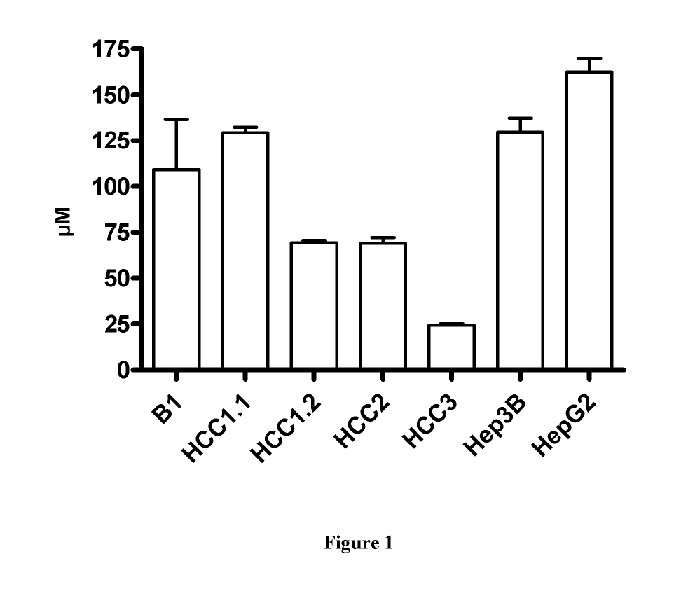

To test the activities of sodium trans-[terachlorobis(1H-indazole)ruthenate(III)] (“drug”), MTT assays were performed using selected hepatocellular carcinoma cell lines. Cells were plated (2×10³ cells in 100 μl/well) in 96-well plates and allowed to recover for 24 hours. The drug was added in another 100 μl growth medium and incubated with cultured cells for 3 hours before the cell culture medium was replaced to remove the drug. Cell death was measured 72 hours after the initial incubation by MTT assay following the manufacturer's recommendations (EZ4U, Biomedica, Vienna, Austria). After 72 hours of treatment, the mean IC₅₀ value was 124.4 μM with HCC1.2, and HCC3 being most sensitive (IC₅₀ values of 62.9 μM and 67.5 μM, respectively) (FIG. 1). Notably, the IC₅₀ values did not correlate with intracellular drug levels determined by inductively-coupled plasma mass spectrometry (ICP-MS) measurements. All tested cell lines revealed similar Ru contents (˜5 ng Ru/105 cells) after 1 hour drug exposure.

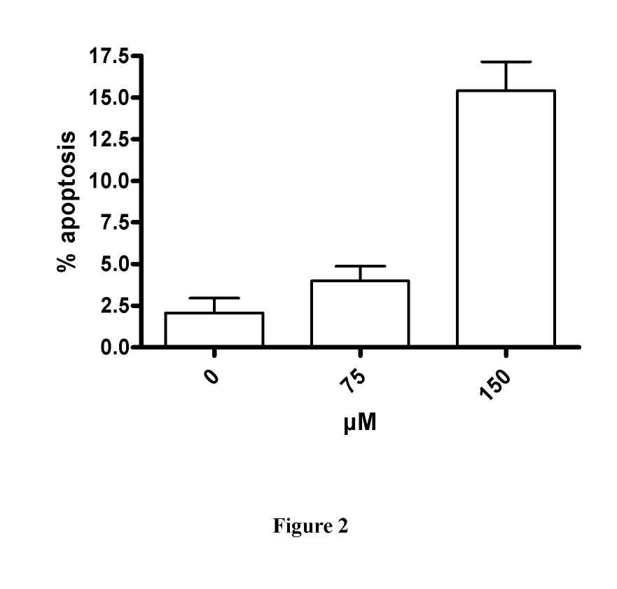

To evaluate the mechanisms underlying the drug activity, apoptosis induction as well as impact on cell cycle distribution and proliferation of Hep3B cells were evaluated (FIG. 2). Specifically, the hepatoma cell line Hep3B was purchased from American Type Culture Collection (ATCC), Manassas, Va. All cells were grown in RPMI 1640 supplemented with 10% FCS. Cultures were regularly checked for Mycoplasma contamination. After 24 hours of drug treatment, cells were harvested, washed in PBS and cytospins were prepared. After fixation with a 1:1 methanol/acetone solution, slides were stained with 4′,6-diamidino-2-phenylindole (DAPI) containing antifade solution (Vector Laboratories, Inc., USA). Nuclear morphology of cells was examined using a Laica DMRXA fluorescence microscope (Laica Mikroskopie and System, Wetzlar, Germany) equipped with appropriate epifluorescence filters and a COHU charge-douples device camera. Duplicate slides were prepared for each cell type/treatment group and 300-500 cells were counted for each sample. Sodium trans-[terachlorobis(1H-indazole)ruthenate(III)] led to significant apoptosis in Hep3B cells.

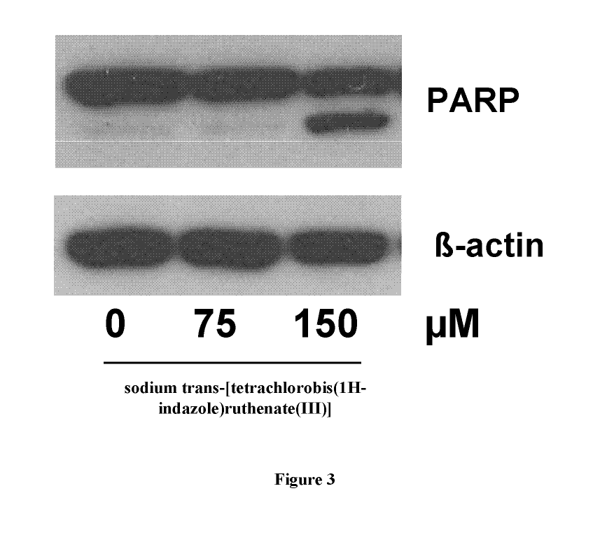

Treatment with sodium trans-[terachlorobis(1H-indazole)ruthenate(III)] led to typical morphological signs of programmed cell death like cell shrinkage, chromatin condensation and formation of apoptotic bodies. Apoptotic cell death induced by sodium trans-[terachlorobis(1H-indazole)ruthenate(III)] was further characterized by PARP cleavage examined by Western Blot Analyses. Specifically, after 24 hours of drug treatment, protein extracts were prepared and Western blot analyses performed. Rabbit Anti-PARP from Apoptosis Sampler kit (Cell Signaling Technology, Beverly, Mass.) (dilution 1:1000)000) was used. For loading control β-actin monoclonal mouse AC-15 (Sigma, USA, dilution: 1:1000) was used. All secondary peroxidase-labeled antibodies were purchased from Santa Cruz Biotechnology and used at working dilution of 1:10000. FIG. 3 shows PARP cleavage induced by sodium trans-[terachlorobis(1H-indazole)ruthenate(III)].

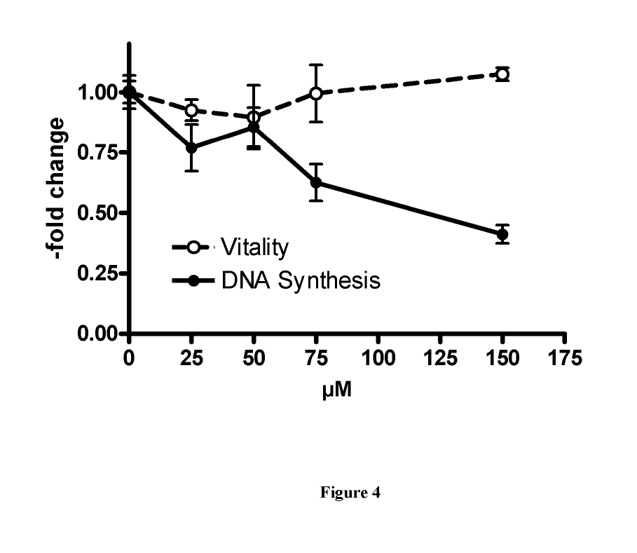

An increase of cells with mitochondrial membrane depolarization (measured by JC-1 staining and FACS analysis) suggested that at least part of the observed apoptotic cell death is induced via the mitochondrial pathway. Furthermore, treatment with sodium trans-[terachlorobis(1H-indazole)ruthenate(III)] was found to significantly reduce DNA synthesis rate measured by ³H-thymidine incorporation (FIG. 4).

With regard to cell cycle distribution, 24 hours of treatment led to increase of cells in G2/M phase (detected by PI-staining followed by FACS analyses). This was accompanied by strong phosphorylation of the stress kinase P38 and the extracellular signal-regulated kinase (ERK), suggesting that P38 might be involved in delayed apoptosis entry.

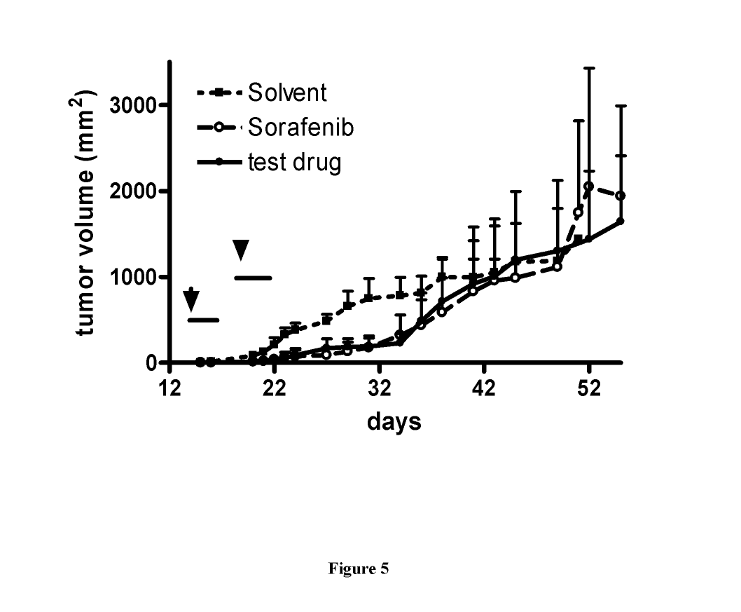

Separately, the activity of sodium trans-[terachlorobis(1H-indazole)ruthenate(III)] against Hep3B cells was evaluated in xenograft experiments using female SCID mice as compared to the standard therapy sorafenib. Sodium trans-[terachlorobis(1H-indazole)ruthenate(III)] was administered W at 30 mg/kg once a week for two weeks. Sorafenib was administered P.O. at 25 mg/kg daily five times a week for two weeks. Sodium trans-[tetrachlorobis(1H-indazole)ruthenate(III)] was well tolerated. In the Hep3B model, sodium trans-[tetrachlorobis(1H-indazole)ruthenate(III)] effectively suppressed the growth of tumor (FIG. 5).

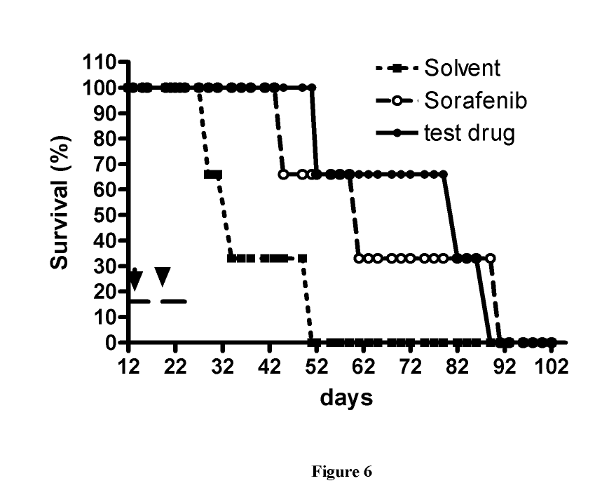

In addition, sodium trans-[terachlorobis(1H-indazole)ruthenate(III)] led to a 2.4-fold increase in life span (mean survival of 80 days as compared to 33 days in control group) and thus was superior to sorafenib monotherapy (1.9-fold survival increase; 60 days as compared to 33 days in the control) (FIG. 6). Taken together, sodium trans-[tetrachlorobis(1H-indazole)ruthenate(III)] is a very promising anticancer drug with significant activity against human hepatocellular carcinoma cell lines in vitro and in vivo.

To determine whether the cytotoxicity of sodium trans-[terachlorobis(1H-indazole)ruthenate(III)] varies in hepatocellular carcinoma cells sensitive and insensitive (or significantly less sensitive) to other drugs, the IC₅₀ values of sorafenib, doxorubicin, and oxaliplatin in various hepatocellular carcinoma cell lines were also determined. In addition, the IC₅₀ values of 5-FU and cisplatin in the Hep3B and HepG2 cell lines were obtained from Kogure et al., Cancer Chemother. Pharmacol., 53(4):296-304 (2004). The ratios of IC₅₀ values of sodium trans-[terachlorobis(1H-indazole)ruthenate(III)] in a cell line sensitive to one of the other drugs and a cell line insensitive to the same drug were calculated. The results are shown in Tables 1-5 below (“Test Drug” in the tables denotes sodium trans-[terachlorobis(1H-indazole)ruthenate(III)]). The data shows that sodium trans-[terachlorobis(1H-indazole)ruthenate(III)] is equally effective in hepatocellular carcinoma cells that are sensitive or resistant to other drugs such as sorafenib, doxorubicin, 5-FU, and platinum agents such as oxaliplatin, and cisplatin. As such, sodium trans-[terachlorobis(1H-indazole)ruthenate(III)] is potentially effective in treating hepatocellular carcinoma resistant to such other drugs.

TABLE 1

| | Test Drug | | Sorafenib | | |
|---|---|---|---|---|---|
| HCC | | IC₅₀ Ratio | | | IC₅₀ Ratio |
| Cell Line | IC₅₀ | (HCC3/B1) | IC₅₀ | | (HCC3/B1) |
| B1 | 83.4 μM | 0.8 | 0.8 | μM | >12.5 |
| HCC3 | 67.5 μM | | >10 | μM | |

TABLE 2

| | Test Drug | | Doxorubicin | | |
|---|---|---|---|---|---|
| HCC | | IC₅₀ Ratio | | | IC₅₀ Ratio |
| Cell Line | IC₅₀ | (HepG2/Hep3B) | IC₅₀ | | (HepG2/Hep3B) |
| Hep3B | 166.9 μM | 0.8 | 69.25 | μM | >3.6 |
| HepG2 | 188.8 μM | | >250 | μM | |

TABLE 3

| | Test Drug | | Oxaliplatin | |
|---|---|---|---|---|
| HCC | | IC₅₀ Ratio | | IC₅₀ Ratio |
| Cell Line | IC₅₀ | (HCC2/HCC3) | IC₅₀ | (HCC2/HCC3) |
| HCC2 | 128.9 μM | 1.9 | 4.05 μM | >9.9 |
| HCC3 | 67.5 μM | | 0.41 μM | |

TABLE 4

| | Test Drug | | Cisplatin | |
|---|---|---|---|---|
| HCC | | IC₅₀ Ratio | | IC₅₀ Ratio |
| Cell Line | IC₅₀ | (HepG2/ Hep3B) | IC₅₀ | (HepG2/Hep3B) |
| Hep3B | 166.9 μM | 1.1 | 3.75 μg/ml | >5.2 |
| HepG2 | 188.8 μM | | 19.4 μg/ml | |

TABLE 4

| | Test Drug | | 5-FU | | |
|---|---|---|---|---|---|
| HCC | | IC₅₀ Ratio | | | IC₅₀ Ratio |
| Cell Line | IC₅₀ | (HepG2/ Hep3B) | IC₅₀ | | (HepG2/Hep3B) |
| Hep3B | 166.9 μM | 1.1 | 227 | μg/ml | >5.2 |
| HepG2 | 188.8 μM | | 17.2 | μg/ml | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method of treating, or delaying the onset of, hepatocellular carcinoma, comprising administering to a patient in need of treatment an effective amount of a compound that is trans-[tetrachlorobis(1H-indazole)ruthenate(III)] or a pharmaceutically acceptable salt thereof.

2. The method of claim 1, wherein the compound is an alkali metal salt of trans-[tetrachlorobis(1H-indazole)ruthenate(III)].

3. The method of claim 1, wherein the compound is sodium trans-[tetrachlorobis(1H-indazole)ruthenate(III)].

4. The method of claim 1, wherein the hepatocellular carcinoma is a refractory or recurrent hepatocellular carcinoma.

5. The method of claim 4, wherein the hepatocellular carcinoma is refractory or resistant to one or more agents chosen from the group consisting of sorafenib, regorafenib, anthracyclines, platinum agents, 5-FU or capecitabine.

6. The method of claim 4, wherein the hepatocellular carcinoma is refractory or resistant to sorafenib.

7. The method of claim 4, wherein the hepatocellular carcinoma is refractory or resistant to doxorubicin.

8. The method of claim 4, wherein the hepatocellular carcinoma is refractory or resistant to oxaliplatin or cisplatin.

9. The method of claim 4, wherein the hepatocellular carcinoma is refractory or resistant to 5-FU.

10. The method of claim 1, further comprising administering to the patient an effective amount of an embolizing agent.

11. A method of treating hepatocellular carcinoma, comprising:
identifying a patient having hepatocellular carcinoma; and
treating the patient with a therapeutically effective amount of a pharmaceutically acceptable salt of trans-[tetrachlorobis(1H-indazole)ruthenate(III)].

12. A kit comprising a unit dosage form of a pharmaceutically acceptable salt of trans-[terachlorobis(1H-indazole)ruthenate(III)] and a unit dosage form of an embolizing agent.
